(19) United States
(12) Patent Application Publication
(10) Pub. No.: US 2011/0064813 A1
(43) Pub. Date: Mar. 17, 2011

(54) USE OF SALSALATE WITH OR WITHOUT CAFFEINE AND WITH OR WITHOUT OMEGA 3, AND OTHER PHARMACEUTICAL COMPOUNDS IN A DISTINCTIVELY UNIQUE NANO-PARTICULATE CAPSULE AND TABLET

(76) Inventor: Charles Vaughn, Champaign, IL (US)

(21) Appl. No.: 12/851,851

(22) Filed: Aug. 6, 2010

Related U.S. Application Data
(60) Provisional application No. 61/243,039, filed on Sep. 16, 2009.

(51) Int. Cl. A61K 31/618; A61K 9/14; A61P 29/00; A61P 35/00; A61P 31/18; A61P 19/02; A61P 9/00; A61P 3/10

(52) U.S. Cl. 424/489; 514/163

(57) ABSTRACT

The invention relates to a compound and administration of the compound to mammals containing salsalate in a nanofied form to reduce inflammation. This compound and administration may be combined with caffeine, omega 3 fatty acids, sodium bicarbonate, and/or simvastatin to further benefit that administration.

### CROSS REFERENCE TO RELATED APPLICATIONS

This application claims the benefit of provisional application 61/243,039 filed Sep. 16, 2009, which is incorporated by reference.

### FIELD OF THE INVENTION

This invention describes a valuable treatment and a distinctive method of delivery for various disease states in which Transcription factor, Nuclear factor-(kappa) B activation, or inflammation arising from yet unidentified sources influences negative health outcomes and is believed to predispose to a host of chronic disease dysfunctions in numerous chronic medical disorders; i.e., arthritis, Type 2 diabetes, obesity and certain cancer tumors. (Nuclear factor-kappa B activation contributes to vascular endothelial dysfunction, Pierce G L et al., Circulation 2009 Mar. 10, 119 (9):1284-92.); Kolima, M. et al., Arthritis Rheum, Jul. 30, 2009 30, 61 (8:1018-1021); Stamatelopoulos K S, Atherosclerosis in Rheumatoid Arthritis vs. Diabetes. A comparative study, Arteriocler Thromb Vasc Biol, July, 2009, Fleischman, A. et al. Salsalate Improves Glycemia and Inflammatory Parameters in Obese Young Adults, Diabetes Care, 2008, February; 31 (2): 289-94).

Salsalate has been utilized for decades as an anti-inflammatory drug. The present invention provides improvement in the administration of salsalate by reducing the particle size through the use of nanofication and micronization.

### DESCRIPTION OF THE DRAWINGS

FIG. 1 is an illustration of a salsate compount.

### BACKGROUND OF THE INVENTION

Type 2 Diabetes has become an epidemic in the past several decades owing to the advancing age of the population, an obesity and decreased physical activity, all of which have been attributed to a Western lifestyle. In the United States almost 8% of the adult population and 19% of the population older than the age of 65 years have diabetes. Several racial and ethnic groups in the United States are at particularly high risk for diabetes, including blacks, Hispanics, Asian and Pacific Islanders and Native Americans (Initial Management of Glycemia in T2Diabetes Mellitus, Nathan, D F, New England Journal of Medicine; Volume 347:1342-49, Oct. 24, 2002, Number 17). In the United States, the estimated cost of providing care for diabetes and its complications is $100 Billion per year, with half the cost attributable to direct care (IBID). These costs strongly demonstrate the need to identify the etiology of Type 2 diabetes and for inexpensive, effective, and safer therapies for the treatment of Type 2 diabetes. To these challenges, current research has identified new pathways playing a major role in the pathogenesis of Type 2 diabetes.

More recently, researchers have proposed, “inflammation participates in the pathogenesis of Type 2 Diabetes (T2D)”. “Weight gain and obesity are accompanied by two inflammatory pathways in adipose tissue and liver, the stress kinase JNK and the Transcription Factor NF-xB which increases the production of pro-inflammatory cytokines and chemokines and promotes the recruitment of macrophages to adipose tissue. Inflammatory mediators induce insulin resistance locally in fat and liver, and in skeletal muscle. The sub-acute chronic inflammation of obesity may therefore provide pharmacological targets for intervention.” (Shoelson et al., J Clin Invest. 2006; Hotamisligl et al., Inflammation and Metabolic Disorders, Nature, 2006; Hasson et al., Inflammation, Atherosclerosis and CAD N Engl J. Med. 2005). Clinical studies in patients presenting with Type 2 Diabetes (T2D) and in obese patients with a pre-disposition of developing Type 2 Diabetes have experienced efficacy in the lowering of glucose levels when taking the tablet forms of salsalate. (Salsalate Improves Glycemia and Inflammatory Parameters in obese young adults, Fleischman, A. et al., Harvard Medical School, Diabetes Care 2008 February; 31(2) 289-94. Epub 2007 Oct. 24), Use of Salsalate to Target Inflammation in the Treatment of Insulin Resistance and Type 2 Diabetes, Goldfine et al., Joslin Diabetes Center and Dept. of Medicine, Harvard Medical School, Clin Trans Sci, 2008 May 1, 1(1): 36-43).

Similarly, reduction in inflammatory events may slow cognitive decline in people with Alzheimer's disease. A study has shown that patients with at least one systemic inflammatory event, such as respiratory or gastrointestinal infections, had twice the rate of cognitive decline. The inflammatory events produced an increase in the pro-inflammatory cytokine tumor necrosis factor (TNF). Reducing the tumor necrosis factor with salsalate may reduce the cognitive decline in Alzheimer's disease.

Administration of salsalate has had several drawbacks. Patients do not like taking the large tablets and some may not be able to swallow the tablets. The dosing regimen is complicated and may confuse some patients. A recommended dosage is often 3000 mg per day. Because the regimen also can take three to four days to obtain efficacy, patients may stop taking the medicine, or fail to associate the benefits with the medicine. During the three to four days, the medicine also provides little relief for the patient.

It is therefore an object of the present invention to provide a salsalate compound and improved delivery method to make it easier to take and obtain short-term, as well as long-term improvements.

### DESCRIPTION OF DRAWINGS

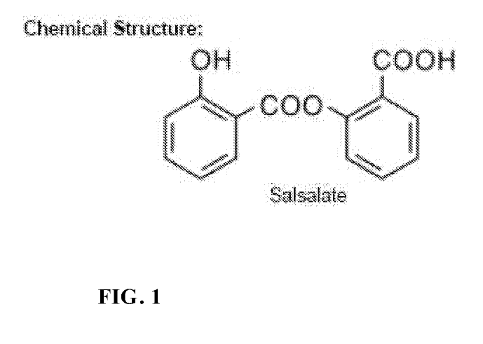

FIG. 1 is the chemical structure of a salsalate.

### DETAILED DESCRIPTION OF THE INVENTION

Distinctive to this invention is the utilization and inclusion of salsalate and other related pharmaceutical compounds to be administered safely, economically, and conveniently utilizing “state of the art” nanotechnology. (Nanotechnology in the emerging Work Package 1—Current Status of Medicinal Nanobiotechnology in Europe, V. Wagner, VDI Technologiezentrum GmbH, 2005.) Salsalate is a non-steroidal anti-inflammatory drug (NSAID). Its Chemical Name is 2-(2-Hydroxybenzoyl)oxybenzoic acid. Its chemical formula is C₁₄14₁₀0₅. This application and claims define salsalate to include salsalate, along with all salts, esters, and functionally equivalent chemical compounds.

To the best of the present inventors knowledge, to date, there are few nanofied prescription products available for the treatment of many common chronic diseases, including the treatment of arthritis, obesity, Type2 diabetes, cancer, hypertension or HIV. Further, those products, currently available by prescription only, are expensive and play host to a robust list of side effects, often life-threatening. It is an object of the present invention to deliver these products via nanotechnology in order to provide more affordable treatments for these chronic disorders while using nanotechnology to reduce the number of side effects when these products are administered in a tablet, capsule or injectable form(s). For example, in the case of Type 2 diabetes patients, treatment and treatment-related side effects cost the U.S. Government over $100 billion per year.

Nanotechnology is emerging as one of the key technologies of the 21st century and is expected to enable developments across a wide range of sectors that can benefit patients and improve their “quality of life”. Scientists define nanotechnology as “the design, characterization, production and application of structures, devices and systems by controlling shape and size at the nanometer scale that is usually set at 1 to 100 nm and nanotechnology makes use of the new properties of materials. Nano biotechnology is the convergence of nanotechnology and biotechnology, and in particular its applications in the medical sector era is considered as one of the most promising and most advanced areas of nanotechnology” (Work Package 1—current status of nanobiotechnology, Work Package 1—Current status of medicinal nanobiotechnology in Europe, V. Wagner, VDI Technologiezentrum GmbH, 2005).

Nanotechnology has numerous applications for health care and is often referred to as “nanomedicine”. Nanomedicine makes advanced drug delivery systems achievable and often results in fewer side effects than when a drug is administered orally as an encapsulated or tablet formulation. Its aim is to improve bioavailability and pharmacokinetics of pharmaceuticals and to replace the more expensive invasive medications by non-invasive routes of administration, for example by administering a pharmaceutical agent sublingually in a nano-particulate spray pump, sublingual fast dissolving tablet or nasally as a spray, the active ingredients are not subject to systemic circulation thereby reducing the number of sides effects. Side effects related to the oral ingestion of NSAIDS are robust and can even cause mortality in certain patients. It is conservatively estimated that “the annual number of hospitalizations in the United States for serious gastrointestinal complications is estimated to be at least 103,000 patients. At an estimated cost of $15,000 to $20,000 per hospitalization, the annual direct costs of such complications exceeds $2 billion” (Wolfe M, MD et al., The New England Journal of Medicine, Jun. 17, 1999, Vol. 340 156 No. 29, pp. 1888-1889)“. Nanoficaton of NSAIDS will reduce this morbidity by administering NSAIDs through less harmful deliver methods, including orally, sublingually, or nasally, via nanoparticulates. This mode of administration would result in achieving high blood levels of the parent compound in a manner of minutes or hours, versus the administration of a tablet or capsule which make require days to achieve therapeutic blood levels. The nano delivery system is a sub-class of advanced delivery systems with a size of micrometer and mostly less than 200 nm. The invention may be composed of one or more of the following delivery systems: Liposome's, Nanosuspensions and/or Polymeric Nanoparticlautes that will be administered sublingual or rapid dissolving tablet or nasal spray.

Salsalate is approved by the United States Food and Drug Administration for the treatment of various types of arthritis. Salsalate has been available as a prescription product for over 40 years and has demonstrated a consistent degree of efficacy with minimal toxicity in thousands of arthritic patients (Salsalate in the Treatment of Rheumatoid Arthritis, a Double Blind Study, vs. Piroxicam, Blanchi, P et al., Journal Internal Medicine, 1989 July-August; 17, 320-03, Longterm Management of Rheumatoid Arthritis with Disalcid, Fazarinc, F. et al., Journal of Internal Medicine, 1980, 8, 339-42). Salsalate is typically administered in large, tablet-sized doses. The maximum dosage is often recommended as approximately 3,000 mgs daily. Salsalate's mode of action as an anti-inflammatory and anti-rheumatic agent may be due to inhibition of synthesis and release of prostaglandins and or its ability to inhibit the transcription of Nuclear Factor Kappa B pathway thereby reducing inflammation or yet some other mechanism yet to be found. In contrast to aspirin, salsalate appears to be safer in long-term administration because it does not contain an Acetyl group, nor does it affect either Cox 1 or 2 inhibition. Salsalate causes less fecal gastrointestinal blood loss than aspirin, and other NSAIDS (Stichtenoth D O, New Non-steroidal Anti-rheumatic Drugs: Selective Inhibitors of Inducible Cyclooxygenase) Med Kim (Munich) 1998 Jul. 15; 93 (7): 407-15). Further, unlike Aspirin, salsalate does not inhibit pro-thrombin time.

Salsalate has shown clinical efficacy equal to diclofenac and causes less gastrointestinal irritation then other NSAIDS (salsalate, a nonacetylated slicylate, is as efficacious as diclofenac in patients in patients with rheumatoid arthritis, Bombardier, C, Journal Rheumatology, 1995 April; 22 (4): 617-24). However, gastric irritation from salsalate has been encountered with the administration of high doses and tinnitus (ringing in the ears) is not infrequent at higher doses. The most common side effects in descending order with salsalate were tinnitus, nausea, hearing impairment, rash and vertigo. Diarrhea and gastrointestinal bleeding have been reported (Physicians Desk Reference, 2009). A major disadvantage of salsalate when administered orally is its slow onset of action “alleviation of symptoms is gradual and full benefit may not be achieved for 3-4 days, when plasma salicylate levels have achieved steady state” (Physicians Desk Reference, 2009).

Many delivery methods would be suitable for the present invention. It is preferred to utilize sublingual sprays, rapid dissolving tablets, and nasal sprays to provide the fastest absorption and reduce gastrointestinal interference. Transdermal, tablet, injection, and other forms may also be used.

Embodiments of this invention include sal salate in nano-particulates delivered through sublingual spray, sublingual rapid dissolving tablet and/or as a nasal spray, or other delivery methods for relief of the signs and symptoms of arthritis, osteoarthritis and rheumatic disorders.

Embodiments of this invention are to achieve greater efficacy of salsalate by including caffeine in the formulation. Caffeine has not been seen to have been combined with salsalate.

This inclusion is logical in that caffeine is generally acknowledged to augment the efficacy effects of analgesics. (Efficacy of Diclofenac With or Without Caffeine: A Randomized, Double-blind Study Crossover Study, Peroutka, S J et al. Headache, 2004 February. 44 (2) 136-41). To facilitate a more rapid onset of action, this invention is distinctive because it calls for salsalate and caffeine to be administered utilizing nano-particulates administered preferably via sublingual spray/rapid dissolving tablet or as a nasal spray. Distinctively, nano-delivery either sublingual or nasally avoids systemic circulation of the drugs resulting in greater bioavailability of drugs resulting in a quicker onset of action and fewer side effects. In addition, the tablet size that is currently approved by the United States Food and Drug Administration is quite large, making it often difficult for patients to swallow and ingest the product ingredients. Of course, this problem affects dosage compliance and efficacy of the clinical benefits of salsalate. Patients who are reluctant to take the tablet or do not see immediate benefits may discontinue taking the proper dosage.

Another embodiment of this invention includes reducing risks of cardiovascular disease in patients with and without Type 2 Diabetes. Specifically, cardiovascular disease is responsible for 65% of deaths in persons with type 2 diabetes.” Reducing cardiovascular disease risk in patients with Type 2 diabetes: a message from the National Diabetes Education Program, Gavin J R et al. Am Family Physician, 2003 Oct. 15, 68(8): 1503-04.1506). Type 2 diabetic patients also have lipid abnormalities (low HDL, and high LDL levels) as well as hypertension. Omega 3 fatty acids have exerted a positive impact on Lipid levels (n3Fatty acids and cardiovascular disease: evidence explained and mechanisms explored, Calder P C; Clinical Science, 2004 July 107-1: 1 through 11); therefore this invention will include the distinctive combination of salsalate with Omega 3 fatty acids.

Another embodiment of this invention is the combination of salsalate nano-particulates to be administered sublingual/spray or sublingual rapid dissolving tablet or nasally, combined with simvaststatin, a prescription lipid lowering agent including the brand ZOCOR by Merck & Co., to be used in dyslipidemic patients with or without diabetes. In the event Type 2 diabetes become refractory to Omega 3 Fatty Acids, simvastatin may be co-administered with salsalate. Simvaststatin is generically as available lipid-lower agent, with extensive documentation of its efficacy for the treatment of dyslipidemia (Efficacy of Statins in Familial Hypeicholesterolemia: A Long Term Study, Versmissen J, et al. British Medical Journal November 11; 337:a2423. doi: 10.1136/bmj.a2423).

Another embodiment of this invention is in the treatment of various forms of cancer and distinctively administered as nano-particulates in the treatment of cancer (Enhancement of Merocyanine 540-mediated Phototherapy by Salicylate, Anderson M S, Cancer Research 53, 806-09, February. 1993). Various studies have demonstrated that Nuclear Factor-kappaB (NF-kappaB) is a common “pathway” in the development of certain tumor types (McCarthy et al. published in the Journal Integr Cancer Ther, 2006; September; 5(3): 252-68 Preadministration Of High-dose Salicylates, Supressors of NF-KappaB Activation May Increase the Chemosensitivity of Many Cancers An Example of Proapoptic Signal Modulation Therapy, Block Center for Integrative Cancer Care—Evanston, Ill., (Preadministration of High Dose Salicylates, Suppressors of NF-kappaB, Integr Cancer Ther, 2006 September, 5(3) 252-68). These investigators have shown that “NF-kappaB activity is elevated in a high proportion of cancers, (The aspirin metabolite salicylate inhibits breast cancer cells growth, Sotiriou, et al., Anticancer Research, ISSN 0250-7005, 1995, 2997-3006 (Sensitization of Neuroblastoma Cells for TRIAL-induced Apoptosis by NF-kappaB Inhibition, (Amman, J U 2009;Mar. 15, 2009. 124. (6): 1301-11) particularly advanced cancers that have been treated previously. Salsalate reduces the up-regulation inasmuch as NF-kappa B promotes. An embodiment of this invention will include distinctive sublingual Nano-particulates to be used as adjuvant therapy in certain tumor sites.

An embodiment of this invention calls for salsalate to be administered as a nano-particulate as adjunctive therapy to correct or diminish HIV-related Endothelial Dysfunction. In a recent clinical trial (S K Gupta, Indiana School of Medicine, The Anti-Inflammatory Agent Salsalate Improves HIV-Related Endothelia Dysfunction; Antiviral Therapy 2007; 12(suppl, 2): L37 abstract nl. P-30) testing verified “salsalate improved endothelial function in HIV-infected patients.” However, a significant number of HIV patients experienced salsalate induced liver dysfunction, limiting the number of patients that could benefit from this therapy. This invention would be administered as Nano—particulates sublingually and or by a nasal spray, thereby bypassing liver metabolism and avoiding hepatoxi city.

In yet another formulation based on particle size reduction, and an enteric coating to afford a rapid dissolution of the drug at the duodenum level. Since salsalate is nearly insoluble in acidic conditions, the disintegration of the drug in the stomach would lead to re-agglomeration of the particles and the benefits of particle size reduction could be lost. Therefore, a formulation containing a buffering agent, such as sodium bicarbonate, could be utilized to raise the stomach pH, reducing the risk of re-agglomeration in the stomach. The inclusion of sodium bicarbonate would increase local effervescence, which boosts the dispersion of the nano-particles. It will also accelerate gastric emptying time, enhancing the transfer of the drug already dissolved or as a nano-dispersion to its preferential absorption site.

An additional embodiment of the invention includes combination with a dosage of a biguanide class of anti-diabetes drug, such as Metformin. This is a standard treatment for Type 2 Diabetes and the includes of the salsalate in the diabetes protocol would have additional benefits.

Use of NSAIDs or salsalate may be effective in the treatment of rheumatoid arthritis and osteoarthritis. Salsalate has been found to be effective and approved for these conditions for over 30 years.

Nanofication of the NSAID or salsalate is preferably incorporated into a nasal spray, but may also be provided by oral suspension, transdermal patches, intravenously, or by other delivery methods. The salsalate compound of the present invention may also be delivered in a capsule or tablet form. Other delivery methods and preparations may be utilized within the scope of the invention.

Pharmacokinetics demonstrate that salicylic acid is the active molecule from the hydrolysis of salsalate in the body. Salsalate is then hydrolyzed by carboxyesterase 2 (HCE2) located in the wall of the intestines. The kinetics of hydrolysis is saturable therefore the release of salsalate should be controlled so as not to over saturate the HCE2 receptors. This unique delivery method will optimize the hydrolysis mechanism and increase the availability of salicylic acid in the small intestine because the concentration of HCE2 in the small intestine is up to 12 times higher than the HCE2 concentration in the colon, thus preventing saturation. Duration of the extended release would be targeted for a maximum of three hours. This would be optimal for the profile for a dual release product wherein one portion of the dose would be released rapidly while the remainder would be slowly released over the next 2-3 hours.

It is preferred to administer the salsalate compound of the present invention in a capsule or tablet form. Other delivery methods and preparations may be utilized.

While particular element, embodiments, and applications of the present invention have been shown and described, the invention is not limited thereto because modifications may be made by those skilled in the art, particularly in light of the foregoing teaching. It is therefore contemplated by the application to cover such modifications and incorporate those features which come within the spirit and scope of the invention.

I claim:

1. A pharmaceutical composition comprising:
a delivery agent,
salsalate particles,
wherein the salsalate particles have a median particle size of about 1 nanometer to about 300 nanometers.

2. The pharmaceutical composition according to claim 1, wherein the composition further comprises caffeine.

3. The pharmaceutical composition according to claim 1, wherein the composition further comprises an omega-3 fatty acid.

4. The pharmaceutical composition according to claim 1, wherein the composition further comprises a buffering agent.

5. The pharmaceutical composition according to claim 1, wherein the composition further comprises sodium bicarbonate.

6. The pharmaceutical composition according to claim 1, wherein the composition further comprises simvastatin.

7. A method of reducing inflammation in mammals comprising administering an effective dosage of salsalate in an nanofied compound.

8. The method of reducing inflammation in mammals according to claim 7, wherein the effective dosage is from about 100 mg to about 3,000 mg per day.

9. The method of reducing inflammation in mammals according to claim 7, wherein the median particle size of the salsalate is between about 1 nanometer and about 300 nanometers.

10. The method of reducing inflammation in mammals according to claim 7, wherein an effective dosage of caffeine is also administered.

11. The method of reducing inflammation in mammals according to claim 7, wherein an effective dosage of omega 3 fatty acid is also administered.

12. The method of reducing inflammation in mammals according to claim 7, wherein a buffering agent is also included in the salsalate compound.

13. The method of reducing inflammation in mammals according to claim 7, wherein an effective dosage of sodium bicarbonate is also administered.

14. The method of reducing inflammation in mammals according to claim 7, wherein an effective dosage of simvastatin is also administered.
